(19) United States
(12) Patent Application Publication
(10) Pub. No.: US 2026/0007847 A1
(43) Pub. Date: Jan. 8, 2026

(54) A FACE MASK WITH A TENSIONER SYSTEM, AND METHODS AND SYSTEMS OF USING THE SAME

(71) Applicant: BESPOKE MEDICAL INNOVATIONS PTY LTD, West Pennant Hills (AU)

(72) Inventor: John Hilton, Baulkham Hills (AU)

(73) Assignee: BESPOKE MEDICAL INNOVATIONS PTY LTD, West Pennant Hills (AU)

(21) Appl. No.: 19/110,891

(22) PCT Filed: Sep. 6, 2023
(86) PCT No.: PCT/AU2023/050862

§ 371 Date: Mar. 12, 2025

(30) Foreign Application Priority Data
Sep. 15, 2022 (AU) 2022902670
May 26, 2023 (AU) 2023901644

(51) Int. Cl. A61M 16/06; A61M 16/20

(52) U.S. Cl. CPC A61M 16/0611; A61M 16/0683; A61M 16/208; A61M 2205/0216

(57) ABSTRACT

A face mask for delivering pressurised gas or air to the mouth and/or nose of a user that has an airbag structure that is configured to isolate dislodging forces experienced by the mask components from being transferred to the air-sealing skin contact regions when in use; and has a tensioner system, external to the airbag, that provides additional sealing pressures to the air-sealing skin contact regions to provide an air seal.

### FIELD

The present disclosure relates generally to devices, systems and/or methods that use a tensioner system to improve the sealing pressures of a face mask against a user's face.

### CROSS-REFERENCE

This application claims priority to Australian Provisional Application No. 2022902670, entitled A FACE MASK WITH A TENSIONER SYSTEM, filed 15 Sep. 2022; and to Australian Provisional Application No. 2023901644, entitled, A FACE MASK WITH A TENSIONER SYSTEM, filed on 26 May 2023. Each of these applications are herein incorporated by reference in their entirety.

### BACKGROUND

Many types of face masks for delivering breathable pressurised gas or air to the nose and/or mouth are commercially available. Continuous Positive Airway Pressure (CPAP) is a common therapy for sleep apnoea sufferers to prevent or reduce the occurrence of sleep apnoea events. CPAP users typically use a nasal, nasal pillow or full-face mask. Nasal and nasal pillow masks deliver pressurised air to the nostrils while full face masks delivery pressurised air to both the nose and mouth.

Some CPAP face mask designs employ a silicone cushion mounted on a rigid support structure held and pressed onto the face by an adjustable head strap system. The silicone cushion engages with the skin around the airways in an attempt to provide an airtight or reasonably airtight seal during sleep. The head straps are adjusted to provide a sealing pressure on the cushion and both the cushion and the skin deform accordingly as the head strap sealing pressure is applied. CPAP face masks typically have elastic head straps with a degree of elasticity or stretchiness that cooperates with the stiffness of cushion's skirt to elastically maintain the position of the mask body. The skirt of the cushion is the sides of the cushion that is not in contact with the skin. The skirt region may vary as the mask moves during use. Overtightening the head straps causes discomfort and may cause pressure sores, for example, on the nasal bridge. Under tightening the head straps results in air leakage with subsequent issues such as noise, irritation of the eyes and/or loss of treatment pressure. Users typically attempt to determine a head strap tension that balances the likelihood of air leakage against the comfort level of wearing the face mask.

Other types of face mask employ a piston-like system whereby the air pressure induces head strap tension as the piston-like mechanism pushes the face contacting component or components and the structure supported by the head straps apart from each other. These face masks may have a generic, typically silicone cushion, face contacting component or a custom-made rigid face contacting component, custom made to fit a specific nose.

Yet other types of face mask have the head strap system attached to the face contacting component or components. A single or multi compartment airbag is attached to the face contacting component(s) to convey air from the air supply tube to openings in the face contacting component(s).

The present disclosure is directed to overcome and/or ameliorate at least one or more of the disadvantages of the prior art, as will become apparent from the discussion herein. The present disclosure also provides other advantages and/or improvements as discussed herein.

### SUMMARY

This summary is not intended to be limiting as to the embodiments disclosed herein and other embodiments are disclosed in this specification. In addition, limitations of one embodiment may be combined with limitations of other embodiments to form additional embodiments.

At least one embodiment is directed to a face mask that is configured to deliver pressurised gas to a user comprising:
an airbag support structure having a flexible airbag support sheet that is connected to one or more head straps;
a supply tube that is configured in use on a user's face to deliver pressurised gas from a flow generator to the face mask and is connected to the flexible airbag support sheet and an airbag, wherein the airbag is configured to induce skin sealing contact pressures from the pressurised air during use;
the airbag is further connected to one or more of the following: a nasal sealing structure of the face mask and a mouth sealing structure of the face mask;
one or more tensioner structures that are configured to augment the skin sealing pressures between the user's air-sealing skin contact regions and the face mask during use and the one or more tensioner structures are external to the airbag;
wherein the one or more tensioner structures consists of two or more elastic tensioner bands or elastic tensioner sheets, attached and stretched between attachment points or lines on the airbag on either side of where the airbag contacts the face and attachment points or lines on either the one or more head straps or the airbag support structure that are distant from the mouth and nearer to the ears;
wherein the airbag is configured in use to permit freedom of movement of the airbag support structure in response to dislodging forces on the face mask; and
wherein the face mask is configured in use to maintain sealing with a user's face and to provide pressurised gas to the user's mouth, the user's nostrils or combinations thereof.

At least one embodiment is directed to a face mask that is configured to deliver pressurised gas to a user comprising:
an airbag support structure that is supported by one or more head straps and provides support to an airbag that is configured to press the airbag against the user's face;
an air supply tube that is configured in use to deliver pressurised gas from a flow generator to the face mask and connects, either directly or indirectly, to the airbag support structure and is supported by the airbag support structure;
the airbag is further connected to one or more of the following: a nasal sealing structure of the face mask and a mouth sealing structure of the face mask;
one or more tensioner structures that are configured to provide in use additional pressure to at least some of the skin sealing contact surfaces in addition to the skin contact pressure provided by the pressurised air;
wherein in use the airbag support structure and the one or more head straps cooperate to allow for movement of the air supply tube in response to dislodging forces;
wherein the airbag support structure and the one or more head straps in use cooperate to provide support to the one or more tensioner structures even as the air supply tube and the airbag support structure move in response to dislodging forces;
wherein the airbag is configured in use to permit freedom of movement of the airbag support structure in response to dislodging forces on the face mask; and
wherein the face mask is configured in use to maintain sealing with a user's face and to provide pressurised gas to the user's mouth, the user's nostrils or combinations thereof.

At least one embodiment is directed to a face mask that is configured to deliver pressurised gas to a user comprising:
an airbag support structure that is supported by the one or more head straps and provides support to an airbag to press it against the user's face whereby the airbag is contained between the support structure and the face;
a flexible air supply tube that in use delivers pressurised gas from a flow generator to the face mask;
the airbag is further connected to one or more of the following: a nasal sealing structure of the face mask and a mouth sealing structure of the face mask;
the one or more head straps induce tension in one or more tensioner structures that in use provides additional pressure to at least some of the skin sealing contact surfaces in addition to the skin contact pressure provided by the pressurised air; and
wherein the face mask is configured in use to maintain sealing with a user's face and to provide pressurised gas to the user's mouth, the user's nostrils or combinations thereof.

At least one embodiment is directed to a face mask that is configured to deliver pressurised gas to a user comprising:
an airbag support structure having a flexible airbag support sheet and one or more head straps;
an air supply tube that is configured to deliver pressurised gas from a flow generator to the face mask and is connected via a rotatable elbow to a flexible airbag support sheet and the airbag;
one or more tensioner structures that are configured to augment the skin sealing pressures between the user's air-sealing skin contact regions and the face mask during use and the one or more tensioner structures are external to the airbag;
wherein the airbag is configured in use to permit freedom of movement of the flexible airbag support structure in response to dislodging forces on the face mask; and
wherein the face mask is configured in use to maintain sealing with a user's face and to provide pressurised gas to the user's mouth, the user's nostrils or combinations thereof.

At least one embodiment is directed to a face mask that is configured to deliver pressurised gas to a user comprising:
an air supply tube, that is configured to provide pressurizes gas to the face mask in use;
a head strap system, that is configured to hold the face mask in place on the user;
an airbag that is attached to the airbag support structure;
an airbag support structure including at least an airbag support sheet;
a tensioner structure;
one or more of the following: a mouth seal and a nasal seal;
wherein the head strap system and the airbag support structure operate as a tension band structure around the head for supporting the pressurised airbag for pressing the airbag against the face; and
wherein the head strap system, the tensioner system and optionally portions of the airbag support structure act as a separate tension band structure around the head for providing additional sealing pressures to the mouth seal and/or the nasal seal.

At least one embodiment is directed to a face mask for delivering pressurised gas or air to the mouth and/or nose. A plenum chamber may be formed to convey pressurised air from a supply tube to the oro-nasal orifices. The plenum chamber may be held towards the face by a support structure attached to the head straps. Pressurised air in the plenum chamber acts to push the support structure away from the face thereby inducing tension in the head straps. The plenum chamber's skirt may be formed from a material lacking the stiffness to provide for an adequate skin sealing pressure of the skin sealing surfaces, thereby decoupling movement of the air supply tube and/or the airbag support structure from the skin sealing surfaces. Substantial movement of the airbag support structure may occur without impacting sealing performance. A tensioner system that provides additional sealing pressures to the air-sealing skin contact regions, the skin sealing pressure resulting from the addition of the plenum chamber's air pressure, pressure resulting from the bellowing action of the airbag plus the tensioner system's pressure. The term bellowing action is used here to describe the effect whereby air pressure against a convex portion of the airbag, such as that facing but not touching the cheeks, induces tension in the airbag, the tension transferring to a concave portion of the airbag, such as that over the mouth area including the mouth band 11 or the mouth seal 18, to induce a sealing pressure to the skin. A venting system provides air washout.

Regarding the sealing of the mouth, the tensioner system may provide a roughly cylindrical or cylindrical-like tension surface over the mouth and adjoining cheeks. As such, tension in the tension surface produces a suitable pressure to the mouth band 11 or the mouth seal 18. Facial anatomies devoid of nasolabial creases can seal adequately when the face contacting surface of the airbag near the mouth is tensioned across the mouth. A C-pull 23 may be used to aid in keeping the mouth opening from collapsing. See, for example, FIGS. 13A and 13B. Many facial anatomies have, for example, nasolabial creases whereby a simple tensioned airbag 4 contact area may leak air. For these anatomies a mouth seal 18 adapts the airbag 4 tension surface to the skin contact anatomy of the face and so conveys the pressure from the tension system to the skin thereby forming an airtight seal. The mouth seal 18 acts as a filler to adapt the facial anatomy to the tensioned portion of the airbag 4 and the tensioner bands 13. The C-pull 23 may be used in conjunction with the mouth seal 18.

At least one embodiment provides an adequate air seal at the skin contact regions around the mouth and/or nostrils. An adequate air seal being an airtight seal, or a substantially airtight seal, where air leakage causes minimal disturbance to the user or their sleeping partner and where the intended CPAP pressure may be maintained by the flow generator for a substantial portion of the therapy for a given night. It is to be understood that an adequate air seal does not exclude an acceptable degree of leakage. Air leakage that disturbs a user or their sleeping partner is air leakage that awakens, or potentially awakens, either of them or causes them to be annoyed. For example, air that blows into the eyes, potentially causing drying of the eye, may be annoying. Another example, may be an unacceptable level of noise.

In certain exemplary embodiments, the airbag's skirt region may be configured to decouple, or substantially decouple, loads and/or movement due to nighttime dislodging forces while in use to permit the skin contacting air sealing surfaces of the face mask to maintain an adequate seal. At least the skirt region of the airbag may be made from a floppy material that does not pass along, or does not substantially pass along, dislodging loads and movements experienced by an air supply tube and an airbag support sheet to the skin contact sealing regions of the face mask. One or more tensioner bands may be supported by one or more head straps and provide additional sealing pressures to the skin sealing surfaces.

In certain embodiments, the tensioner bands may be attached to one or more of the following; an airbag, one or more stiffener arms, the head strap system, upper cheek buckles, lower cheek buckles, outer points on the airbag support and combinations thereof. In certain embodiments, the tensioner sheets may be attached to one or more of the following; an airbag, one or more stiffener arms, the head strap system, upper cheek buckles, lower cheek buckles, outer points on the airbag support and combinations thereof. In certain embodiments, the tensioner bands may be attached at points. In other embodiments, the tensioner sheets may be attached in points or lines or combinations thereof. The tensioner attachment support points and/or lines remain relatively stable as dislodging loads are experienced by the face mask. The head straps and the airbag support structure cooperate to provide relatively stable support to the tensioner attachment support points and/or lines and dislodging loads and/or movements of the face mask are largely isolated from the tensioner support points and/or lines.

### BRIEF DESCRIPTION OF THE DRAWINGS

Exemplary embodiments will now be described, by way of example only, with reference to the accompanying drawings.

FIG. 1 illustrates a face mask device, according to at least one embodiment.

FIG. 2A illustrates a head strap system 7 and the airbag support 3 with the upper head strap 8 detached shown in FIG. 1.

FIG. 2B illustrates a hidden line view of a portion of FIG. 2A and shows how the airbag support sheet may attach to the head strap system as shown in FIG. 1.

FIGS. 3A-3D illustrates four views of the nasal seal 14 from FIG. 1.

FIGS. 4A-4B are two views of the non-inflated airbag 4 showing its shape and the various holes, according to at least one embodiment.

FIG. 5 illustrates the mouth band 11, tensioner bands 13 and sealing bumps 17, according to at least one embodiment.

FIG. 6A illustrates the nasal seal 14 with an attached mouth seal 18, according to at least one embodiment.

FIG. 6B illustrates the nasal seal 14 with a rope-like mouth seal 18 around the mouth on a person, according to at least one embodiment.

FIG. 6C illustrates the nasal seal 14 and mouth seal 18 of FIG. 6A on a user.

FIG. 7 illustrates an embodiment with stiffener arms 6 and broad tensioner sheets 20, according to at least one embodiment.

FIG. 8 is a cross sectional view of the embodiment of FIG. 7.

FIG. 9 illustrates the broad tensioner sheets 20 and tensioner bands 13 affixed to the airbag 4, according to at least one embodiment.

FIGS. 10A-10D are four views of the nasal seal 14 having tensioner attachments 24, according to at least one embodiment.

FIG. 11 illustrates a nasal-only embodiment on a user, according to at least one embodiment.

FIG. 12 illustrates the airbag 4 with an attached mouth seal 18 constructed with an EVA sheet, a pair of mouth tensioner bands 13 and a pair of nasal tensioner bands 13, according to at least one embodiment.

FIG. 13A illustrates the airbag 4 with two C-pulls 23 in two pockets sewn onto the airbag 4 and a pair of mouth tensioner bands 13 and a pair of nasal tensioner bands 13, according to at least one embodiment.

FIG. 13B illustrates a C-pull illustrated in FIG. 13A.

FIG. 14A illustrates an embodiment without stiffener arms 6 and with a generic silicone nasal seal 14, according to at least one embodiment.

FIG. 14B illustrates the back of the head of the embodiment of FIG. 14A.

FIG. 15 is an exploded view of at least one embodiment.

FIG. 16 is a top view of FIG. 15.

FIGS. 17A-17B illustrate a silicone nasal seal 14.

FIGS. 18A-18B illustrates a rigid nasal seal 14.

FIG. 19A illustrates a hidden line view of a silicone airbag 4 with integrated mouth seal 18, nasal seal 14 and broad tensioner sheets 20, according to at least one embodiment.

FIG. 19B is a rear view of FIG. 19A.

FIG. 19C is a perspective view of FIG. 19A.

### DETAILED DESCRIPTION

The following description is provided in relation to several embodiments that may share common characteristics and features. It is to be understood that one or more features of one embodiment may be combined with one or more features of other embodiments. In addition, a single feature or combination of features in certain of the embodiments may constitute additional embodiments. Specific structural and functional details disclosed herein are not to be interpreted as limiting, but merely as a representative basis for teaching one skilled in the art to variously employ the disclosed embodiments and variations of those embodiments.

The subject headings used in the detailed description are included only for the ease of reference of the reader and should not be used to limit the subject matter found throughout the disclosure or the claims. The subject headings should not be used in construing the scope of the claims or the claim limitations.

The term “substantially” as used herein means that at least 80%, 85%, 95%, 96%, 97%, 98%, or 99%, of the items, properties and/or functions referred to.

The term “each” as used herein means that at least 95%, 96%, 97%, 98%, 99% or 100% of the items, properties and/or functions referred to perform as indicated.

As used herein, the term skirt means that part of the plenum chamber (i.e., that part of an airbag) lacking the stiffness to provide for an adequate skin sealing pressure of the skin sealing surfaces and thereby decoupling movement of the airbag support structure from the skin sealing surfaces. The skirt region of the airbag is bounded by the outer contact region or regions of the airbag support structure and the outer contact region or regions of the skin, nasal seal and/or mouth seal of the airbag.

As used herein, the term floppy means a soft and easily bent material that maintains its structure and yet independently lacks the ability to maintain a firm shape. For example, a fabric material that droops under gravity but still maintains its woven structure. Another example would be a thin silicone sheet that would also droop under gravity.

Certain embodiments of this disclosure may be useful in several areas. For example, one or more of the following non-limiting exemplary applications: adult, paediatric and neonatal breathing circuits as used in hospitals and medical care units; dust, chemical and biological filtering breathing systems as used in industrial environments or on battlefields, underwater breathing and pilot breathing masks. For example, a dust filtering device would have ambient air drawn or pushed through a filter and then provided to the face mask.

As used herein, the term airtight seal means a seal that has little to no leakage or, where the air leak is small enough, that the flow generator may maintain the required air pressure and where the air leak sound level is below 50 dB(A) at 1 m.

To provide an airtight seal, or substantially airtight seal, against the skin may be accomplished by using a skin contact pressure that is higher than the plenum chamber's air pressure otherwise the air may leak. Many commercial masks employ a silicone cushion having a skin contact portion or portions with corresponding skirt portion or portions that are supported by a mask body. A head strap system pulls the mask body towards the face and the mask body applies pressure to the skirt portion which conveys this extra pressure to the skin contact portion which, in addition, to the internal plenum chamber air pressure, provides a skin contact pressure that is higher than the plenum chamber's air pressure. The skirt therefore requires sufficient stiffness to convey this extra pressure from the mask body to the skin sealing surfaces. Conversely, the skirt requires flexibility to allow for movement of the mask body due to night-time dislodging forces without dislodging the skin sealing surfaces. The flexibility seeks to decouple, as far as possible, movement of the mask body from movement of the skin sealing surfaces. Stiffness and flexibility are opposing requirements and so designers develop designs that seek to provide an optimal balance between stiffness and flexibility to provide a comfortable and adaptable mask that is not prone to air leaks arising from typical night-time dislodging forces.

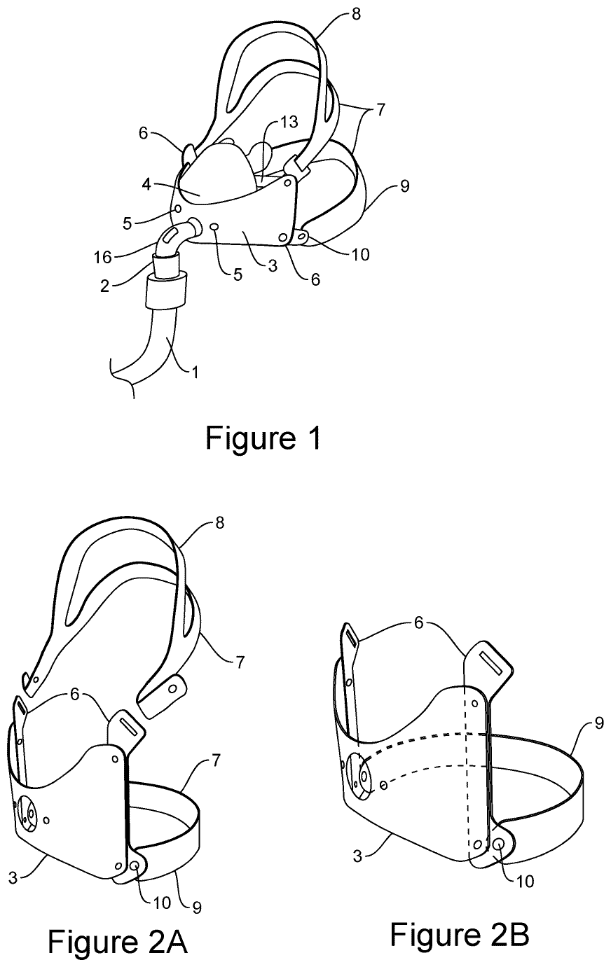

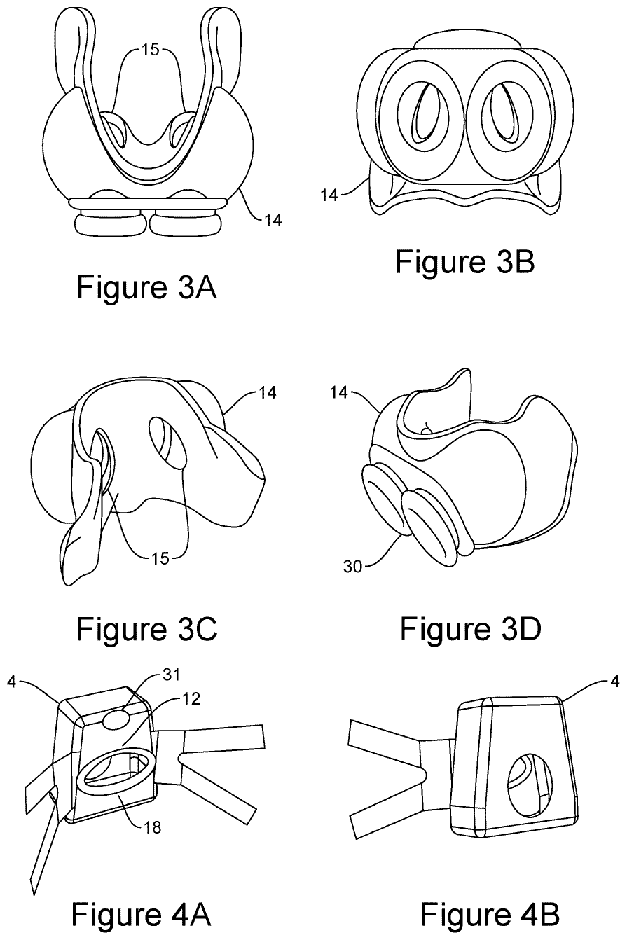

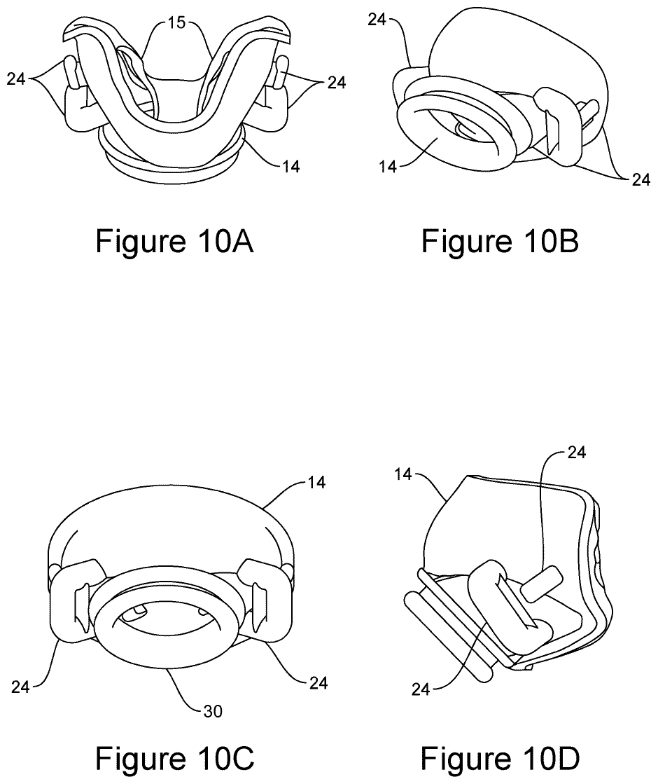

The exemplary embodiment shown in FIGS. 1, 2A and 2B has a flexible air supply tube 1 attached to a rotatable elbow 2 that connects to a flexible airbag support 3 and an airbag 4. In certain embodiments, the elbow may be rotatable, partially rotatable, or not rotatable. In certain embodiments, the air supply tube may be flexible, partially flexible or not flexible. In certain embodiments, the airbag support 3 may be a sheet, one or more sheets, a net, or combinations thereof. Two air vents 5 may be assembled into coinciding holes in the airbag 4 and the airbag support 3. The sides of the airbag support 3 may be attached to stiffener arms 6 which may be part of a head strap system 7. Upper and lower head straps 8 and 9 attach to the two stiffener arms 6. Clip-on attachments 10 may be used for attaching the lower head strap 9 to provide for donning and doffing of the face mask. FIG. 5 shows the mouth band 11 having a mouth hole 12 for breathing. The mouth band 11 may be affixed to the airbag 4. In the exemplary embodiment, a tensioner system consisting of elastic tensioner bands 13 connect the mouth band 11 to the stiffener arms 6. A rigid nasal seal 14, as illustrated in FIGS. 3A-3D, may be attached to the airbag 4 having corresponding hole or holes for receiving the airbag interface feature 30. The nasal seal 14 shown in FIGS. 3A-3D interfaces to the airbag 4 for conveying air between the nostril holes 15 and the airbag 4. Another exemplary nasal interface 14 embodiment is shown in FIGS. 10A-10D having a single oval airbag interface feature 30. An anti-asphyxia valve 16 may be integrated into the rotatable elbow 2 as shown in FIG. 1. Optional sealing bumps 17 may be attached to the mouth band 11 where required to fill in indentations in a person's face, commonly found either side of the mouth, that would otherwise allow air to leak.

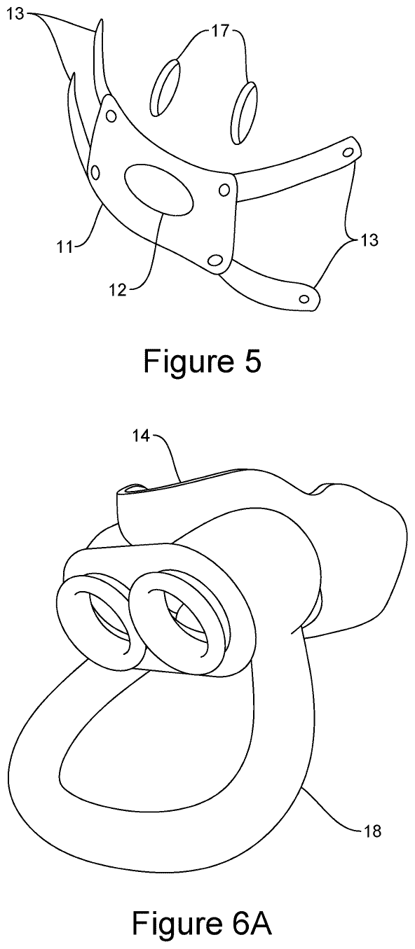

FIGS. 4A and 4B show a flexible airbag 4 (for example a floppy airbag) with a mouth hole 12, a single nasal attachment opening 31, a mouth seal 18 and an attached tensioner system. The nasal seal 14, as shown in FIGS. 10A-10D and having a single oval airbag attachment feature 30, is attached to the airbag 4 by pressing the airbag interface feature 30 into the nasal attachment hole 31.

The air supply tube 1 conveys pressurised air from a flow generator to the mask. The rotatable elbow 2 allows for free movement of the air supply tube 1 and the anti-asphyxia valve 16 opens to allow breathing of air whenever there is little or no pressure in the elbow, for example, during a power blackout. Pressurised air inflates the floppy airbag 4 causing it to push the airbag support 3 and the mouth band 11 away from each other thereby inducing tension in the head straps 8 and 9. The airbag's skirt region is configured to decouple, or substantially decouple, loads and/or movement due to nighttime dislodging forces while in use to permit the skin contacting air sealing surfaces of the face mask to maintain an adequate seal. At least the skirt region of the airbag 4 is made from a floppy material that does not pass along, or does not substantially pass along, dislodging loads and movements experienced by the air supply tube 1 and the airbag support sheet 3 to the skin contact sealing regions of the face mask. Tensioner bands 13 are supported by the head straps 7 and provide additional sealing pressures to the skin sealing surfaces. The two air vents 5 expire air to allow for the replenishment of fresh air from the flow generator. The design of the upper airbag may be such that air pressure pushes the nasal seal 14 and the nearby airbag in such a way that the induced skin contact pressure may be higher than the pressurised air pressure thus providing an airtight, or substantially airtight, seal.

When dislodging forces are experienced by the air supply tube 1 or other mask components, the airbag support 3 may displace and/or deform to accommodate these dislodging forces. The airbag support 3 cooperates with the head strap system 7 to transfer the dislodging forces to the head in a way that avoids dislodging the sealing surfaces near the mouth and nostrils. A pillow pressing against the support sheet 3 may collapse the underlying airbag allowing pressures from the pillow to pass through to the cheek. The overall effect is that the mouth band 11 and the nasal seal 14 are sufficiently isolated from the dislodging forces which would otherwise dislodge these skin contact regions and potentially cause, or cause, an air leak.

In at least one embodiment, a flexible airbag is an airbag constructed from materials that allow for freedom of movement of the airbag support structure in relation to the skin contacting surfaces without transferring, or without substantially transferring, loads that may disturb an adequate air seal of the skin contacting surfaces. Examples of materials include a thin silicone membrane, plastic films such as polyethylene film, nonbreathing fabrics such as oilcloth and/or combinations thereof.

In at least one embodiment, a flexible airbag support is an airbag support that may be constructed from a sheet of material that bends under the typical loads experienced by a mask. The flexible airbag support may transfer tension loads when pulled but may buckle under compressive loads. Examples of materials include polypropylene sheet that is a 160-micron thick, a nylon net, cotton fabric and/or combinations thereof.

In at least one embodiment, the head straps 8 and 9 may be made with a non-elastic material, for example denim, to provide a less compliant head strap system 7 that acts as an anchor for the airbag support 3 and so reduces the likelihood of air leaks as a result of dislodging forces experienced during the night.

In this exemplary embodiment, the nasal seal 14 may be designed to account for the substructure of the face to avoid uncomfortable and potentially injury causing pressure points on the skin. The airbag facing side of the nasal seal 14 may be designed to engage with the airbag 4 in a way that provides a balanced pressing of the nasal seal 14 that seeks to provide an even skin pressure of the rigid skin contact surface with the skin. The nasal seal 14 has two integrated round protrusions that fit into corresponding holes in the airbag 4 thereby attaching the nasal seal 14 to the airbag 4. Another attachment employs an elliptical or oval hole in the airbag into which a corresponding rigid oval protrusion having a lip that may be pressed through the hole with an adjoining channel to capture the edge of the oval hole. Many alternative designs are possible for attaching the nasal seal 14 to the airbag 4 while allowing for disassembly, if so desired, for the purpose of cleaning the parts. For example, a plastic receptacle feature may be heat bonded or glued to a fabric airbag for receiving a corresponding snap-in protrusion feature of the nasal seal 14.

The exemplary embodiment shown in FIG. 7 has the airbag support 3 attached on either side to stiffener arms 6. Two broad tensioner sheets 20 are attached on either side to the airbag 4 behind the mouth seal 18 and a loop sewn into the outer ends fits over the stiffener arms 6. A rigid nasal seal 14 is attached to the airbag 4 allowing for pressurised air to freely pass between the airbag's plenum chamber and the nostril holes 15.

In at least one embodiment, the airbag support 3 may be non-elastic, i.e., not stretchable, or substantially not stretchable, although it may be flexible and bend, for example, in response to dislodging forces on the air supply tube 1 being conveyed to the airbag support 3. In other embodiments, the airbag support 3 may have elasticity although this may result in it expanding and contracting as the air pressure increases and decreases.

In at least one embodiment, the airbag 4 may be non-elastic, or substantially non-elastic, yet collapsible when pressed with a pressure that overcomes the pressurised air. In other embodiments, the airbag 4 may have a degree of elasticity although this may result in the airbag 4 expanding and contracting in response the changes in the air pressure. In certain embodiments, the airbag may have an elasticity in which case it may change size as the air pressure changes. In certain embodiments, the airbag skirt may be made from a non-elastic material that maintains, or substantially maintains, a size as the air pressure varies. The airbag skirt may be made from a material with a degree of elasticity provided the resulting change in size due to changing air pressure continues to permit the face mask to provide an adequate seal.

The sealing bumps 17 modify the shape of the mouth band 11 to account for faces with groove-like indentations, commonly found on either side of the mouth. Pressing a simple tape-like tensioned mouth band 11 over these indentations may result in gaps through which air may leak. In at least one embodiment, the design of the mouth band 11 may be derived from an analysis of a scan of the 3D surface shape of the person's face.

In at least one embodiment, the mask provides a comfortable-to-wear face mask that lacks pain-inducing pressure points and provides consistent skin contact pressure regions that surround the mouth and nostril openings.

In at least one embodiment, the upper and lower head straps 8 and 9 on the back of the head and on the neck are positioned a suitable distant from each other to provide a wide base to withstand tugs, up and down relative to an upright head, on the air supply tube 1. As the air supply tube 1 may be pulled in an upwards direction the load may be transferred to and resisted by the back of the neck. Likewise, as the air supply tube 1 is pulled in a downwards direction the load is transferred to and resisted by the upper back and/or top of the head.

A suitable material may be chosen for at least the skin contacting regions of mouth band 11 to feel comfortable and suitably handle leakage of mouth liquids. For example, suitable material may include, but not limited to, one or more of the following: silicone, cotton, cotton/elastane blend, silk, linen, polyurethane, neoprene, thermoplastic elastomer, and Santoprene® thermoplastic vulcanizates (TPV).

In at least one embodiment, the nasal seal 14 may be 3D printed from a suitable biocompatible material, for example, an acrylonitrile butadiene styrene (ABS) and may be surface finished to provide a hygienic surface that does not accumulate material to avoid bacterial growth. Other suitable materials may be used, for example, polylactic acid (PLA), nylon, polyethylene (PE), polymethyl methacrylate (PMMA), polysulphone, polyphosphaszene, polydimethylsiloxane, thermoplastic elastomer, or combinations thereof. The material may have antibacterial properties and/or additives.

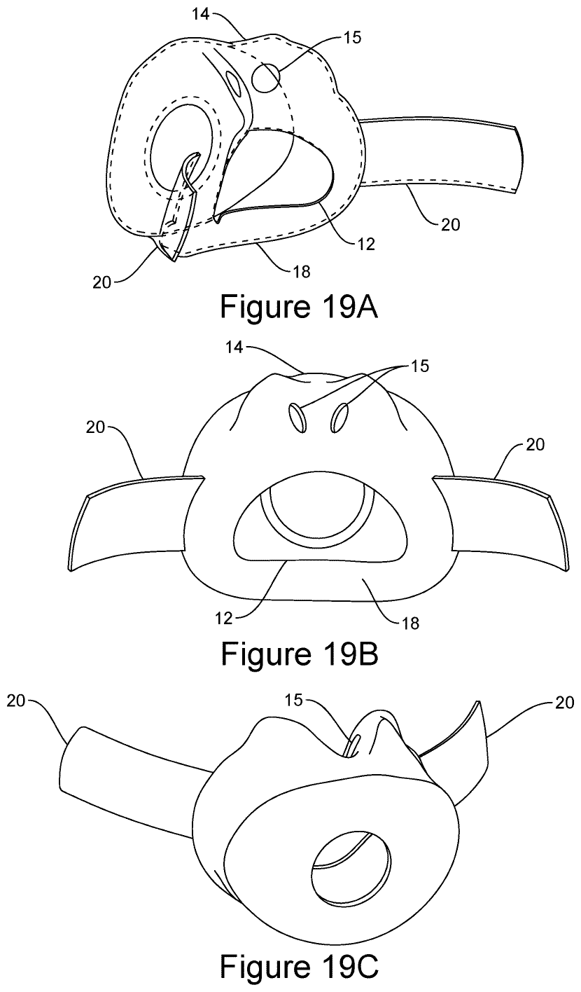

The airbag 4, mouth band 11 and tensioner bands 13 may be integrated into one component as, for example, a moulded silicone part as shown in FIGS. 19A-19C.

In at least one embodiment, the tensioner system may be two flexible and broad tensioner sheets 20 attaching either side of the mouth band 11 to each of the corresponding stiffener arms 6 as shown in FIG. 7.

In another exemplary embodiment, the nasal seal 14 is a flexible nasal interface, for example, a nasal interface, known as a nasal cradle, provides a silicone surface that presses against the skin under the nose, along the upper lip, into the nasolabial folds, against the cheeks and sometimes over the ala at the sides of the nose. There are either two nostril holes or a single hole allowing the nostrils to breath.

In another exemplary embodiment, the tensioner bands 13 and mouth band 11 are integrated as a single elastic sheet with a mouth hole, the airbag 4 being suitably affixed to said elastic sheet. For example, the items may have their mating surfaces glued within a rectangular shape as shown in FIG. 9.

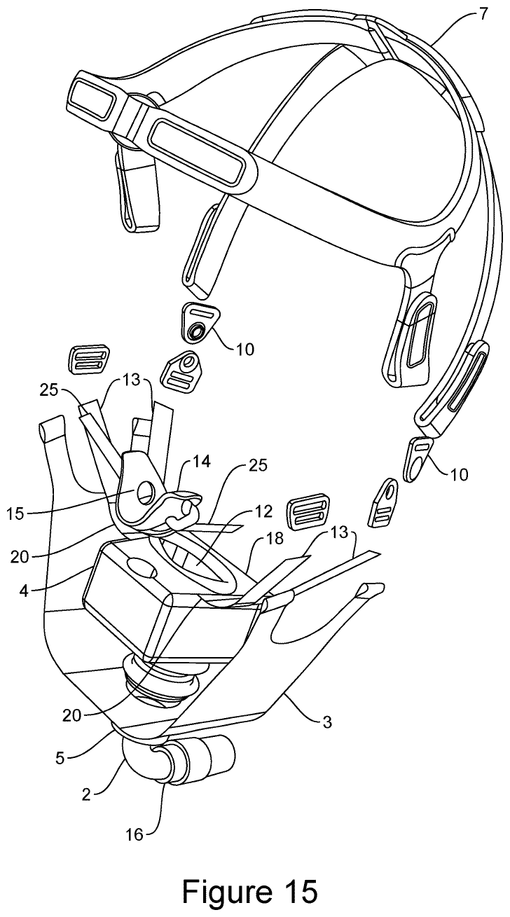

In another exemplary embodiment, a tensioner system may be used to aid the sealing of the nasal seal 14 using the design of the tensioner system employed for the mouth band 11. FIGS. 10A-10D shows a nasal seal 14 having tensioner attachments 24. FIG. 15 shows an embodiment of a face mask having the additional nasal tensioners 25. In certain embodiments, the additional sealing pressure provided by the bellowing effect of the airbag provides an adequate nares seal. In other embodiments, a tensioner system provides the required additional sealing pressure. The tensioner attachments 24 are positioned to provide a balanced loading to the nasal seal 14 such that an axis may be identified allowing rotation of the nasal seal 14 about this axis to find a balance whereby the skin sealing pressures higher on the nose and lower on the upper lip may be perceived by the user as being the same, substantially the same, or similar pressures.

The tensioners used for the nasal region and/or the mouth region may be tensioner bands, tensioner sheets, or combinations thereof. For example, FIG. 9 shows two mouth tensioner sheets 20 combined with four mouth tensioner bands 13. In certain embodiments, the tensioner bands may be attached to one or more of the following; the airbag 4, the stiffener arms 6, the head strap system 7, the upper cheek buckles 26, the lower cheek buckles 27, outer points on the airbag support 3 and combinations thereof. In certain embodiments, the tensioner sheets may be attached to one or more of the following; the airbag 4, the stiffener arms 6, the head strap system 7, the upper cheek buckles 26, the lower cheek buckles 27, outer points on the airbag support 3 and combinations thereof. In certain embodiments, the tensioner bands 13 may be attached at points. In other embodiments, the tensioner sheets 20 may be attached in points or lines or combinations thereof. The tensioner attachment support points and/or lines remain relatively stable as dislodging loads are experienced by the face mask. The head straps and the airbag support structure cooperate to provide relatively stable support to the tensioner attachment support points and/or lines and dislodging loads and/or movements of the face mask are largely isolated from the tensioner support points and/or lines.

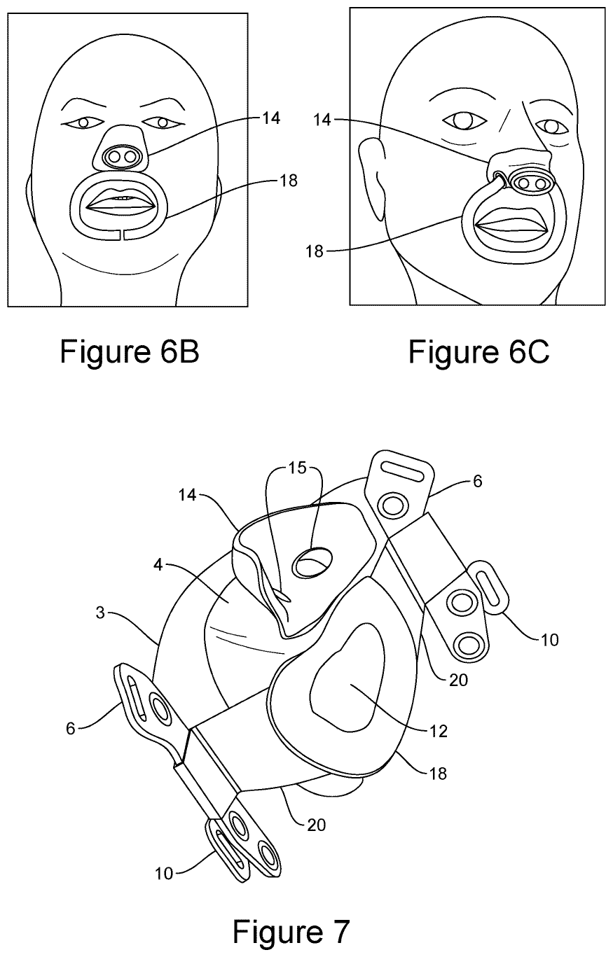

In another exemplary embodiment, there may be a flexible rope-like or tube-like mouth seal 18, shown in FIG. 6, that may be pressed against the skin around the mouth by the mouth band 11 has the effect of lifting the airbag 4 (for example a floppy airbag) and/or mouth band 11 away from the skin in the vicinity of the mouth seal 18. The mouth seal 18 may encircle the mouth and nostrils or encircle the mouth except for the nasal area where it is affixed to the nasal seal 14 to form an adequate air seal. When the mouth seal 18 is affixed to the nasal seal 14 it may or may not be attached to the airbag 4. The tube-like structure may be flexible to conform to the shape of the face, allowing for facial movement by flexing accordingly. The mouth seal 18 enhances the air sealing characteristic by increasing the skin contact pressure where the mouth seal 18 may be in contact with the skin due to the mouth seal 18 holding the airbag 4 and/or mouth band 11 away from the skin.

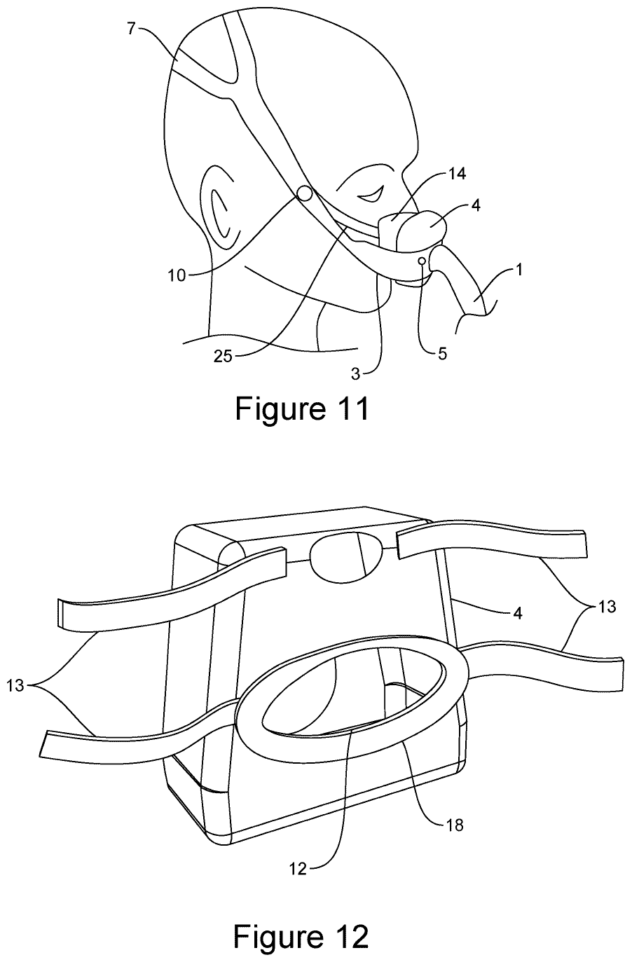

In another exemplary embodiment as shown in FIG. 11, the mouth band 11 and associated items are omitted, the nasal seal 14 being aided by a tensioner system. The mouth may not be covered. The mask has a nasal seal 14, airbag 4, tensioner bands 13, airbag support structure 3, air supply tube 1, air vents 5 and head strap system 7. The airbag 4 may be a conduit that conducts pressurised air between the air supply tube 1 interface and the nares. The pressurised air applies a skin contact sealing pressure to the skin sealing surfaces both through direct compression on the airbag and/or the nasal interface that may be directly over the skin contact regions and due to the billowing effects of the airbag. Additional skin contact sealing pressure may be provided by the tensioner bands which are supported by the head straps. Dislodging loads pulling down towards the chin cause the airbag support structure to press against the upper lip and/or the mouth area and so provide a countering response to the downwards load. The flexibility of the airbag skirt region provides for movement that provides for an adequate air seal at the skin contact region.

In another exemplary embodiment, the head strap system 7 does not include stiffener arms 6. Exemplary embodiments may employ suitable head strap systems. The tensioner bands 13 or tensioner sheets 20 may be attached to the head strap system 7, or to outer points on the airbag support 3. Movement of the attachment points of the tensioner bands 13 or tensioner sheets 20 results in changes in length and therefore changes in tension in the tensioner bands 13 or tensioner sheets 20 which, in turn, may induce movement, or insubstantial movement, of the mouth seal 18 whereby movement of the mouth seal 18 is less than the movement of the tensioner attachment points or lines. The effect of the tensioner system may be to decouple movement, or substantially decouple movement, of the tensioner attachment points from the mouth band 11 and/or the nasal seal 14.

Air vent placement and design may vary. Exemplary embodiments may employ suitable type of air vent designs.

In another exemplary embodiment, a mouth seal 18 consisting of a layer of 2 mm thick ethylene-vinyl acetate (EVA) foam sheet onto which a suitably shaped foam filler 22 is attached. The EVA foam may be pressed by the braided elastic tensioner bands 13 and this pressure may then be applied more evenly across the mouth by the EVA foam sheet, than if the elastic tensioner bands 13 were used without the EVA foam sheet. A fabric layer may be stitched over the top of the EVA foam sheet and the filler to provide an aesthetic and/or comfortable skin contact surface.

In another exemplary embodiment, the tensioner system may comprise a pair of stretchable, or substantially stretchable, fabric tensioner sheets 20 such as 95% cotton 5% elastane (also called Spandex® or Lycra®) which is comfortable against the skin and the increased width, compared with thinner braided elastic tensioner bands 13, provides a lighter skin pressure for a given tensioning load. Each tensioner sheet 20 may be affixed to the airbag 4 by stitching its whole edge adjacent to the mouth opening to provide a more even, or substantially even, tension load across the tensioner sheet's 20 width compared to attaching at the corners of the tensioner sheet 20 to corresponding points on the airbag 4. Each tensioner sheet 20 may also be attached to a stiffener arm 6 by stitching a channel at the outer edges of the tensioner sheets 20 and sliding the stiffener arms 6 into these channels. Other attachment methods may also be used including, for example, designs providing adjustment of the operational length of the tensioner sheet 20.

Tensioner sheets 20 may be attached close to each side of the airbag mouth hole 12 to provide support for the mouth seal 18. The tensioner sheets 20 and the tensioned portion of the airbag 4 form a tension sheet whereby the curvature of this tension sheet 20 determines the pressure normal to the sheet as is understood from shell theory known in the mechanical engineering arts. The mouth seal 18 acts as a filler that carries the pressure provided by the tension sheet, along with the air pressure provided by the pressurised air in the airbag 4, to the skin in seeking to apply an even, or a substantially even, pressure around the mouth opening, or mouth and nasal openings, that is higher than the pressurised air pressure for providing an airtight, or substantially airtight, seal.

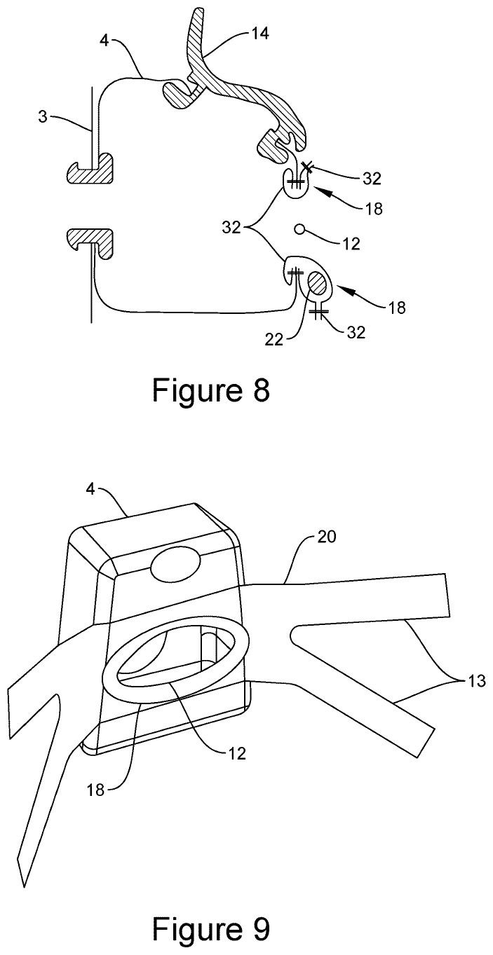

In an exemplary embodiment as shown in FIG. 8, the mouth seal 18 may be formed by stitching the Lycra fabric, as used for the tensioner sheets 20, to both sides of the fabric airbag 4 then cutting the mouth opening and pulling the inside Lycra fabric through the opening. A cylindrical foam filler 22 with suitably tapered ends to provide clearance near the philtrum may be stitched between the two pieces of Lycra fabric to form the mouth seal 18 in seeking to avoid potentially fraying fabric edges in the mouth opening. The Lycra fabric may be then trimmed near to this final stitch. Stitchings 32 are illustrated in FIG. 8 in cross section by short lines to show how the airbag 4 fabric and the two mouth seal 18 Lycra fabric pieces may be stitched together to contain the foam filler 22. The foam filler 22 may have other suitable shapes for the purpose of conveying pressure from the tension sheet to the skin as facial shapes vary from person to person. The teeth and/or gums inside the mouth provide support for the flesh being pressed by the mouth seal 18. The flexibility of the mouth seal 18 allows the shape of the mouth seal 18 to tend to follow the opening and closing of the mouth.

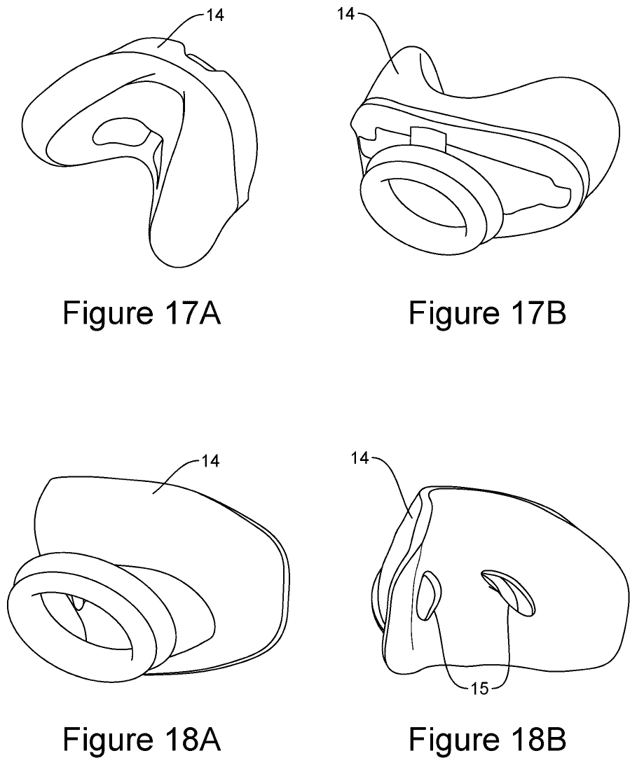

In another exemplary embodiment, a nasal seal 14, as shown in FIGS. 17A and 17B, has a skin contacting region made from a material that may comfortably conform to a user's nose to provide an adequate airtight seal and an airbag interfacing region that may be rigid and that has a feature designed to attach to the airbag's 4 nasal attachment hole 31. For example, the nasal seal 14 may be formed by assembling a nasal cushion to a rigid interfacing component that includes the airbag attachment feature 30. The nasal cushion may be an existing silicone nasal cradle that is common in the industry.

In another exemplary embodiment, a rigid nasal seal 14, as shown in FIGS. 18A and 18B, has an integrated airbag interface feature 30 allowing for pressurised air to be conveyed between the airbag and the nostrils.

In another exemplary embodiment, the mouth seal consists of a doughnut shaped bag-like membrane encompassing a fluid or a gel. The fluid ensures even pressure may be applied across the surface of the membrane and so the pressure from the tension sheet is conveyed evenly, or substantially evenly, to the skin. A gel may also provide a suitable pressure transference although, if the membrane is damaged or ruptured, a gel does not present a troublesome spill or leak as a fluid would.

In another exemplary embodiment the mouth seal may be formed and attached by moulding a silicone, or silicone-like, material through many small holes or slots encircling the mouth hole 12 in the airbag 4. Other suitable mouth seal mould shapes may also be used.

Other suitable designs for the tensioner sheets 20 and the mouth seal 18 are contemplated.

In another exemplary embodiment, the head straps may be made from non-elastic, or substantially non-elastic, material. It is common practice to use elastic head straps to provide compliance to the mask. In certain embodiments, the elastic head straps may be omitted as the airbag 4 and tensioners provide elastic compliance to the mask. Non-elastic head straps improve performance as to air leaks by reducing the movement of the airbag support 3 in response to night-time dislodging forces.

In another exemplary embodiment, the stiffener arms 6 may be integrated into stiff sections of the airbag support 3. Stiffeners and/or stiffener sections may further be added along the airbag support 3 to help it to retain a ruled surface shape as it curves around the front of the face.

In another exemplary embodiment, the oro-nasal interface may be provided by a silicone cushion similar in shape to traditional cradle oro-nasal silicone cushions and supported by the airbag 4 with the oro-nasal cushion feature and the airbag being moulded as a single component as shown in FIGS. 19A-19C or where there is a suitable attachment between the airbag 4 and said silicone cushion.

In another exemplary embodiment, the tensioner bands 13 may be attached to the airbag 4 further away from the face to pull the sides of the airbag 4 towards the face to aid the bellowing of the airbag 4 and so provide additional sealing pressure around the mouth and/or nasal openings.

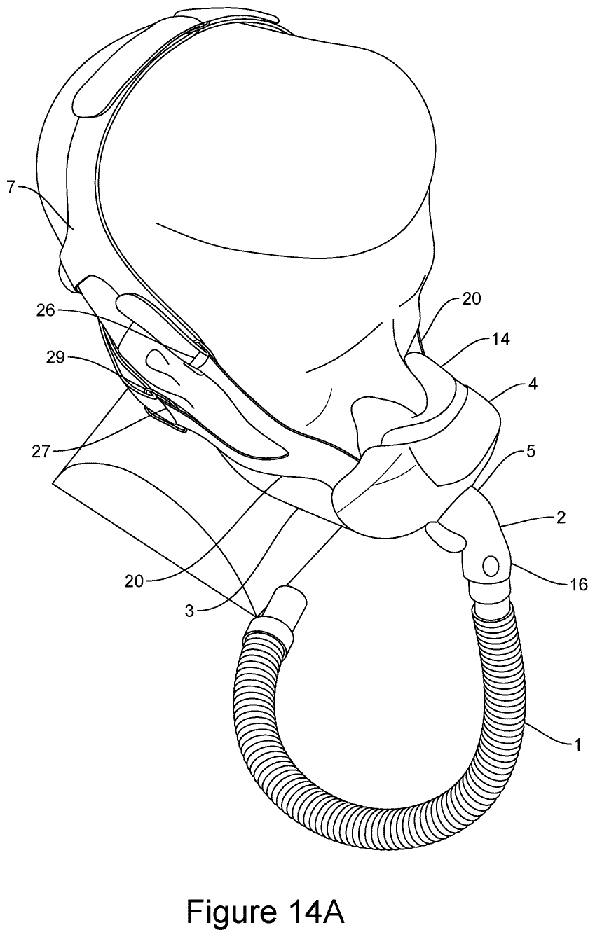

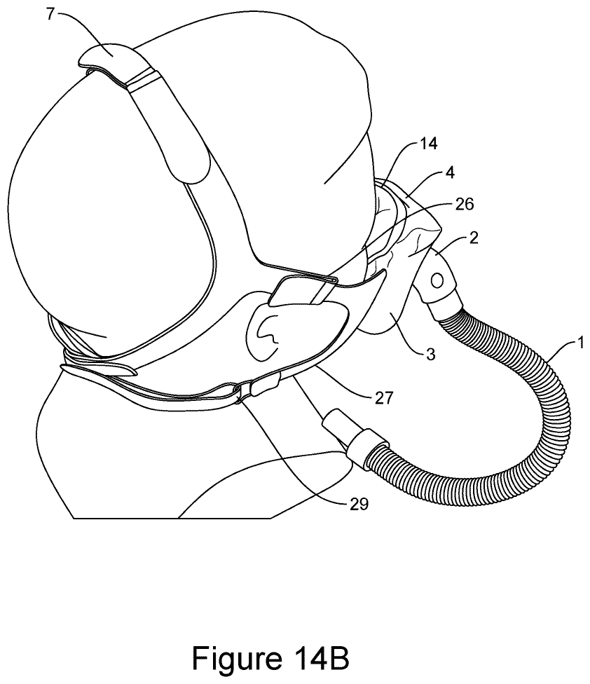

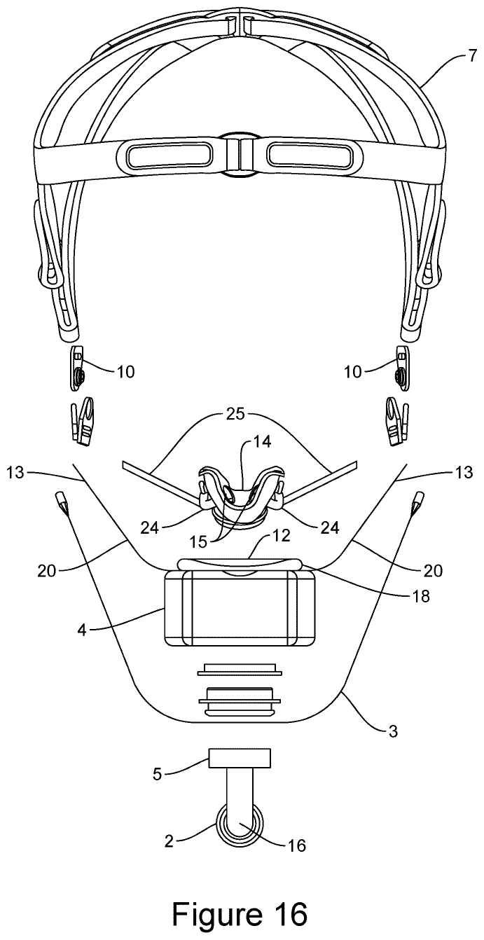

In another exemplary embodiment, as shown in FIGS. 14A, 14B, 15 and 16 the airbag 4 may be constructed from a fabric that is airtight, or substantially airtight, and sewn to form the encompassing shape, the mouth seal 18 may be formed by sewing an elastic material over a soft foam form providing the shape as shown in FIGS. 12 and 15. A tube attachment 19 is held in a hole in the airbag 4 with a press fitted ring 21 located inside the airbag 4. The press fitted ring 21 is shown outside the airbag in the exploded view of FIG. 15. The elbow 2 attaches to the tube attachment with a latching mechanism to allow for quick attachment and detachment. The hole in the airbag support 3 may be press fitted onto the tube attachment 19. The nasal seal 14 may be press fitted into the corresponding oval hole in the airbag 4. The four ends of the airbag support 3 may be affixed to two upper cheek buckles 26 and two lower cheek buckles 27. The airbag support 3 may be made of a polyethylene sheet that has each end threaded through its respective rectangular hole in the cheek buckles then heat bonded using a bag sealer. To minimize facial marking the broad tensioner sheets 20 and/or the tensioner bands 13 may be fabricated from Lycra material and folded and stitched along the middle using a zigzag stitch so the folded edges stay smooth against the face. The inner ends of the mouth tensioner sheets 20 may be stitched with a straight line to the fabric airbag 4 on either side of the mouth seal. Each nasal tensioner 25 may be threaded through the nasal tensioner attachments 24 on either side of the nasal seal 14 and a slot cut in the nasal tensioner 25 fitted over the locking protrusion 28 on the nasal seal 14. The four ends of the mouth tensioner bands 13 and the two ends of the nasal tensioners 25 may be threaded together through the respective far rectangular holes then threaded back through the neighbouring rectangular hole then stitched to form a loop over the bar between the rectangular holes. The upper head strap 8 ends may be threaded into the corresponding holes in the upper cheek buckles 26. The cheek snaps 29 may be threaded onto the ends of the lower straps of the head strap system 7.

FIG. 12 illustrates the airbag 4 of an embodiment having a mouth seal 12 constructed with a layer of 2 mm thick EVA foam that may be stitched along with a layer of Lycra onto the airbag 4 around the mouth hole 15. The EVA foam may provide enough body to the mouth seal to maintain its shape when not in contact with the face yet allow it to form a shape over the mouth region, adapting to the anatomy, to provide an adequate airtight seal. A suitably shaped cylindrical foam filler 22 may be then stitched between the EVA and Lycra layers with the Lycra stretched during the sewing to firmly hold the foam filler 22 in place and to avoid folds and creases of the Lycra in the skin contact area. Two mouth tensioner bands 13 may be sewn to the airbag behind the EVA to deliver a concentrated pressure to the EVA foam which in turns may deliver a more even pressure to the mouth via the cylindrical foam filler 22 and the Lycra layer.

In FIG. 12, two nasal tensioner bands 13 are also shown which pull the nasal attachment hole towards the face thereby adding skin sealing pressure to the nasal seal 14, in addition to the skin sealing pressure due to the pressurised air pushing on the airbag 4 which then pushes on the nasal seal 14.

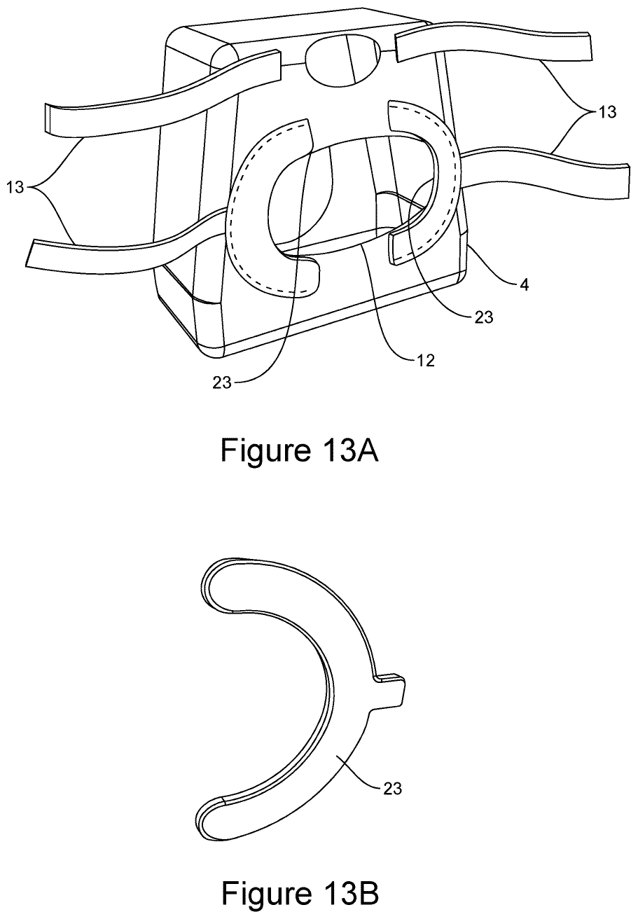

In another exemplary embodiment, as shown in FIG. 13A, the airbag 4 has two pockets sewn on either side of the mouth hole 12 for receiving a C-pull 23. A C-pull is shown in FIG. 13B. C-pulls 23 may be fitted into loops formed at the ends of the tensioner bands 13 and the tensioner bands 13 may be threaded through gaps in the pocket stitching such that the C-pull may be pulled into place in the pocket by tension in the tensioner band 13. This style of mouth interface may be used, for example, with facial anatomies absent of deep nasolabial creases on either side of the mouth.

In another exemplary embodiment, as illustrated in FIGS. 19A-19C, the airbag 4, the mouth seal 18, the nasal seal 14, the tensioner bands 13 and/or tensioner sheets 20 may be moulded as a single item. In other embodiments, one or more of these components and/or other components not listed herein, may be attached rather than being moulded by a suitable attachment mechanics such a gluing. In this example, skin contact sealing pressures may be applied due to the pressurised air by the direct pressure on the back of the skin sealing surfaces and by a billowing effect from the shape of the airbag. The skirt region of the airbag may be floppy, or substantially floppy, whereby the sealing pressures provided by the degree of stiffness in the skirt may be insufficient to produce, in addition to the sealing pressures arising from the pressurised air, an adequate air seal at the skin contacting regions. Additional skin sealing pressures may be provided by tension in the tensioner sheets 20, the tensioner sheets 20 being supported at their outer extremities by attachment, either directly or indirectly, to the head strap system 7. In other embodiments, one or more tensioner bands, one or more tensioner sheets, or combinations thereof may provide the additional skin sealing pressures. The head strap system 7 provides support to the airbag support sheet 3 and to the support attachment lines and/or points of the tensioner sheets 20. Elasticity of the tensioner sheets 20 may provide a degree of decoupling of movement of the support attachment lines and/or points from the skin contact sealing regions while providing additional skin contact sealing pressures. The head strap system 7 may provide a degree of stability to the support attachment lines and/or points. The airbag support structure provides a supporting layer over the face and the tensioner structure provides a separate tensioning layer over the face. Movement may occur to the face mask due to dislodging forces experienced during use and yet the skin contact sealing regions may be isolated, or substantially isolated, from the dislodging movements of the face mask thereby maintaining an adequate air seal.

In another exemplary embodiment, the integrated airbag of the previous paragraph is adapted to attach to and replace the silicone cushion of an existing face mask. The opening that is distal from the face may receive pressurised air. Attachment features may be provided to attach the extremities of the tensioner sheets 20, either directly or indirectly, to the head strap system.

The use of tensioners permits the stiffness of at least the skirt to be reduced while maintaining or improving the air sealing performance of the face mask. In certain embodiments, the at least the skirt may have a degree of stiffness to transfer pressures from the airbag support structure to increase the skin contact sealing pressures. For example, silicone masks shown in FIGS. 19A-19C may be modified such that their skirts are stiffer and may not be characterised as floppy but are more akin to a traditional silicone cushion that is modified in production to introduce a reduction in stiffness. In such and exemplary cushion, the skin contact sealing pressures are from one or more of the following: (i) the pressurised air pressing on internal surfaces that are directly above the corresponding skin contact regions, (ii) pressure transferred due to the stiffness of the skirt as a compressive stress within the skirt material, (iii) pressures arising from a billowing action whereby the pressurised air induces tensions in convex portions of the airbag that result in pressures being applied via concave portions of the airbag, such as the airbag regions over the skin contact regions, to the skin contact regions and (iv) pressures arising from tension in the tensioners applying pressures via concave portions, such as the airbag regions over the skin contact regions, to the skin contact regions. Using the approach illustrated in this exemplary embodiment, face masks may be produced that have a reduction in stiffness, as compared to traditional silicone face masks cushions, and yet may produce a more comfortable fit of the mask to the face with good sealing characteristics. In other words, using this exemplary embodiment, a face mask may be produced that has excellent sealing characteristics but with only a minimum reduction in stiffness of the silicone cushion by using one or more tensioners to enhance the sealing characteristics of the face mask by absorbing dislodging forces without disrupting the sealing characteristics of the face mask.

Further advantages and/or features of the claimed subject matter will become apparent from the following examples describing certain embodiments of the claimed subject matter.

1. A face mask that is configured to deliver pressurised gas to a user comprising:
an airbag support structure having a flexible airbag support sheet that is connected to one or more head straps;
a supply tube that is configured in use on a user's face to deliver pressurised gas from a flow generator to the face mask and is connected to the flexible airbag support sheet and an airbag, wherein the airbag is configured to induce skin sealing contact pressures from the pressurised air during use;
the airbag is further connected to one or more of the following: a nasal sealing structure of the face mask and a mouth sealing structure of the face mask;
one or more tensioner structures that are configured to augment the skin sealing pressures between the user's air-sealing skin contact regions and the face mask during use and the one or more tensioner structures are external to the airbag;
wherein the one or more tensioner structures consists of two or more elastic tensioner bands or elastic tensioner sheets, attached and stretched between attachment points or lines on the airbag on either side of where the airbag contacts the face and attachment points or lines on either the one or more head straps or the airbag support structure;
wherein the airbag is configured in use to permit freedom of movement of the airbag support structure in response to dislodging forces on the face mask; and
wherein the face mask is configured in use to maintain sealing with a user's face and to provide pressurised gas to the user's mouth, the user's nostrils or combinations thereof.
   2. A face mask that is configured to deliver pressurised gas to a user comprising:
an airbag support structure that is supported by one or more head straps and provides support to an airbag that is configured to press the airbag against the user's face;
an air supply tube that is configured in use to deliver pressurised gas from a flow generator to the face mask and connects, either directly or indirectly, to the airbag support structure and is supported by the airbag support structure;
the airbag is further connected to one or more of the following: a nasal sealing structure of the face mask and a mouth sealing structure of the face mask;
one or more tensioner structures that are configured to provide in use additional pressure to at least some of the skin sealing contact surfaces in addition to the skin contact pressure provided by the pressurised air;
wherein in use the airbag support structure and the one or more head straps cooperate to allow for movement of the air supply tube in response to dislodging forces;
wherein the airbag support structure and the one or more head straps in use cooperate to provide support to the one or more tensioner structures even as the air supply tube and the airbag support structure move in response to dislodging forces;
wherein the airbag is configured in use to permit freedom of movement of the airbag support structure in response to dislodging forces on the face mask; and
wherein the face mask is configured in use to maintain sealing with a user's face and to provide pressurised gas to the user's mouth, the user's nostrils or combinations thereof.
   3. A face mask that is configured to deliver pressurised gas to a user comprising:
an airbag support structure that is supported by the one or more head straps and provides support to an airbag to press it against the user's face whereby the airbag is contained between the support structure and the face;
a flexible air supply tube that in use delivers pressurised gas from a flow generator to the face mask;
the airbag is further connected to one or more of the following: a nasal sealing structure of the face mask and a mouth sealing structure of the face mask;
the one or more head straps induce tension in one or more tensioner structures that in use provides additional pressure to at least some of the skin sealing contact surfaces in addition to the skin contact pressure provided by the pressurised air; and
wherein the face mask is configured in use to maintain sealing with a user's face and to provide pressurised gas to the user's mouth, the user's nostrils or combinations thereof.
   4. A face mask that is configured to deliver pressurised gas to a user comprising:
an airbag support structure having a flexible airbag support sheet and one or more head straps;
an air supply tube that is configured to deliver pressurised gas from a flow generator to the face mask and is connected via a rotatable elbow to a flexible airbag support sheet and the airbag;
one or more tensioner structures that are configured to augment the skin sealing pressures between the user's air-sealing skin contact regions and the face mask during use and the one or more tensioner structures are external to the airbag;
wherein the airbag is configured in use to permit freedom of movement of the flexible airbag support structure in response to dislodging forces on the face mask; and
wherein the face mask is configured in use to maintain sealing with a user's face and to provide pressurised gas to the user's mouth, the user's nostrils or combinations thereof.
   5. The face mask of any of examples 1 to 4, wherein the one or more tensioner structures consists of two or more elastic tensioner bands or elastic tensioner sheets, attached and stretched between points on the airbag on either side of where the airbag contacts the face and points on either the one or more head straps or the airbag support structure that are distant from the mouth and nearer to the ears.
   6. The face mask of any of examples 1 to 5, wherein the airbag support structure is configured such that in use the airbag is substantially decoupled from movement of the airbag support structure from the user's facial contact surfaces.
   7. The face mask of any of examples 1 to 6, wherein the one or more head straps are substantially non-elastic.
   8. The face mask of any of examples 1 to 7, wherein the airbag is configured to fit between a portion of the user's face and the flexible airbag support sheet and provides pressurised gas to the nose and mouth of the user while allowing movement of the flexible airbag support sheet without substantially transferring the movement to the user's facial contact surfaces.
   9. The face mask of any of examples 1 to 8, wherein the flexible airbag support sheet further includes one or more cheek stiffeners that are configured to minimise movement when dislodging loads are experienced by the air supply tube and the flexible airbag support sheet.
   10. The face mask of any of examples 1 to 9, wherein the airbag is configured in use to form a substantially airtight seal with the user's face.
   11. The face mask of any of examples 1 to 10, wherein the airbag is configured in to use to have minimal air leakage with the user's face in contact with the mouth seal, nasal seal and/or the airbag.
   12. The face mask of any of examples 1 to 11, wherein the airbag is configured in use to provide an adequate air seal at the skin contact regions around the mouth and/or nostrils of the user.
   13. The face mask of any of examples 1 to 12, wherein the face mask is configured such that in use pressurised air inflates the flexible airbag causing the airbag to push the flexible airbag support sheet and a mouth band away from each other thereby inducing tension in the one or more head straps.
   14. The face mask of any of examples 1 to 13, wherein the flexible airbag support sheet cooperates with the one or more head straps to transfer the dislodging forces to the user's head in a way that avoids dislodging the sealing surfaces near the mouth and/or nostrils.
   15. The face mask of any of examples 1 to 14, wherein the flexible airbag support sheet is non-elastic or substantially non-elastic.
   16. The face mask of any of examples 1 to 15, wherein the flexible airbag support sheet is elastic.
   17. The face mask of any of examples 1 to 16, wherein the airbag is non-elastic.
   18. The face mask of any of examples 1 to 17, wherein the airbag is elastic.
   19. The face mask of any of examples 1 to 18, wherein the airbag has a skirt region and the skirt region of the airbag is made from a floppy material that does not pass, or does not substantially pass, dislodging loads and movements experienced by the end of the supply tube to the skin contact sealing regions.
   20. The face mask of any of examples 1 to 19, wherein the one or more tensioner structures are two flexible and broad tensioner sheets attaching on either side of the mouth band to each of the corresponding stiffener arms.
   21. The face mask of any of examples 1 to 20, wherein a face seal structure is a flexible rope-like structure that is configured to be encircle the user's mouth, the user's nostrils or combinations thereof.
   22. The face mask of any of examples 1 to 21, wherein the airbag is constructed from a fabric that is airtight, or substantially airtight.
   23. The face mask of any of examples 1 to 22, wherein the airbag is configured to billow upon the addition of pressurised gas from the flow generator.
   24. The face mask of any of examples 1 to 23, wherein the airbag is flexible.
   25. The face mask of any of examples 1 to 24, wherein the one or more tensioner structures have one or more tensioner support points or lines that are configured to remain relatively stable as dislodging loads are experienced by the face mask.
   26. The face mask of any of examples 1 to 25, wherein the one or more head straps and/or the airbag support structure are configured to cooperate in use to provide relatively stable support to the one or more tensioner structures whereby dislodging loads and movements of the face mask are largely isolated from the one or more tensioner support structures.
   27. The face mask of any of examples 1 to 25, wherein the one or more tensioner structures is attached to one or more of the following; an airbag, one or more stiffener arms, the head strap system, upper cheek buckles, lower cheek buckles, outer points on the airbag support and combinations thereof.
   28. The face mask of any of examples 1 to 25, wherein the one or more tensioner structures are one or more tensioner sheets that are attached to one or more of the following; an airbag, one or more stiffener arms, the head strap system, upper cheek buckles, lower cheek buckles, outer points on the airbag support and combinations thereof.
   29. The face mask of any of examples 1 to 28, wherein the airbag is moulded with an integrated mouth seal and a nasal seal, tensioner bands and/or tensioner sheets that are one or more of the following: attached to the airbag, an integral part of the moulded airbag, or combinations thereof.
   30. A face mask that is configured to deliver pressurised gas to a user comprising:
an air supply tube, that is configured to provide pressurizes gas to the face mask in use;
a head strap system, that is configured to hold the face mask in place on the user;
an airbag that is attached to the airbag support structure;
an airbag support structure including at least an airbag support sheet;
a tensioner structure;
one or more of the following: a mouth seal and a nasal seal;
wherein the head strap system and the airbag support structure operate as a tension band structure around the head for supporting the pressurised airbag for pressing the airbag against the face; and
wherein the head strap system, the tensioner system and optionally portions of the airbag support structure act as a separate tension band structure around the head for providing additional sealing pressures to the mouth seal and/or the nasal seal.
   31. The face mask of example 30 where the tension band structure substantially decouples, or decouples, movement of the face mask due to dislodging forces experienced during use from the skin contact sealing regions to provide an adequate airtight seal.
   32. The face mask of examples 30 or 31, wherein the one or more tensioner structures consists of two or more elastic tensioner bands or elastic tensioner sheets, attached and stretched between points on the airbag on either side of where the airbag contacts the face and points on either the one or more head straps or the airbag support structure that are distant from the mouth and nearer to the ears.
   33. The face mask of any of examples 30 to 32, wherein the airbag support structure is configured such that in use the airbag is substantially decoupled from movement of the airbag support structure from the user's facial contact surfaces.
   34. The face mask of any of examples 30 to 33, wherein the one or more head straps are substantially non-elastic.
   35. The face mask of any of examples 30 to 34, wherein the airbag is configured to fit between a portion of the user's face and the flexible airbag support sheet and provides pressurised gas to the nose and mouth of the user while allowing movement of the flexible airbag support sheet without substantially transferring the movement to the user's facial contact surfaces.
   36. The face mask of any of examples 30 to 35, wherein the flexible airbag support sheet further includes one or more cheek stiffeners that are configured to minimise movement when dislodging loads are experienced by the air supply tube and the flexible airbag support sheet.
   37. The face mask of any of examples 30 to 36, wherein the airbag is configured in use to form a substantially airtight seal with the user's face.
   38. The face mask of any of examples 30 to 37, wherein the airbag is configured in to use to have minimal air leakage with the user's face in contact with the mouth seal, nasal seal and/or the airbag.
   39. The face mask of any of examples 30 to 38, wherein the airbag is configured in use to provide an adequate air seal at the skin contact regions around the mouth and/or nostrils of the user.
   40. The face mask of any of examples 30 to 39, wherein the face mask is configured such that in use pressurised air inflates the flexible airbag causing the airbag to push the flexible airbag support sheet and a mouth band away from each other thereby inducing tension in the one or more head straps.
   41. The face mask of any of examples 30 to 40, wherein the flexible airbag support sheet cooperates with the one or more head straps to transfer the dislodging forces to the user's head in a way that avoids dislodging the sealing surfaces near the mouth and/or nostrils.
   42. The face mask of any of examples 30 to 41, wherein the flexible airbag support sheet is non-elastic or substantially non-elastic.
   43. The face mask of any of examples 30 to 42, wherein the flexible airbag support sheet is elastic.
   44. The face mask of any of examples 30 to 43, wherein the airbag is non-elastic.
   45. The face mask of any of examples 30 to 44, wherein the airbag is elastic.
   46. The face mask of any of examples 30 to 45, wherein the airbag has a skirt region and the skirt region of the airbag is made from a floppy material that does not pass, or does not substantially pass, dislodging loads and movements experienced by the end of the supply tube to the skin contact sealing regions.
   47. The face mask of any of examples 30 to46, wherein the airbag is configured to billow upon the addition of pressurised gas from the flow generator.
   48. The face mask of any of examples 30 to 47, wherein the airbag is flexible.
   49. The face mask of any of examples 30 to 48, wherein the one or more tensioner structures have one or more tensioner support points or lines that are configured to remain relatively stable as dislodging loads are experienced by the face mask.
   50. The face mask of any of examples 30 to 49, wherein the one or more head straps and/or the airbag support structure are configured to cooperate in use to provide relatively stable support to the one or more tensioner structures whereby dislodging loads and movements of the face mask are largely isolated from the one or more tensioner support structures.
   51. The face mask of any of examples 30 to 50, wherein the airbag is moulded with an integrated mouth seal and a nasal seal, tensioner bands and/or tensioner sheets that are one or more of the following: attached to the airbag, an integral part of the moulded airbag, or combinations thereof.
   52. A method of using a face mask using any of the face masks disclosed in examples 1 to 51.

Any description of prior art documents herein, or statements herein derived from or based on those documents, is not an admission that the documents or derived statements are part of the common general knowledge of the relevant art.

While certain embodiments have been shown and described herein, it will be obvious to those skilled in the art that such embodiments are provided by way of example only.

In the foregoing description of certain embodiments, specific terminology has been resorted to for the sake of clarity. However, the disclosure is not intended to be limited to the specific terms so selected, and it is to be understood that a specific term includes other technical equivalents which operate in a similar manner to accomplish a similar technical purpose. Terms such as “left” and right”, “front” and “rear”, “above” and “below” and the like are used as words of convenience to provide reference points and are not to be construed as limiting terms.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

It is to be understood that the present disclosure is not limited to the disclosed embodiments and is intended to cover various modifications and equivalent arrangements included within the spirit and scope of the present disclosure. Also, the various embodiments described above may be implemented in conjunction with other embodiments, e.g., aspects of one embodiment may be combined with aspects of another embodiment to realize yet other embodiments. Further, independent features of a given embodiment may constitute an additional embodiment. In addition, a single feature or combination of features in certain of the embodiments may constitute additional embodiments. Specific structural and functional details disclosed herein are not to be interpreted as limiting, but merely as a representative basis for teaching one skilled in the art to variously employ the disclosed embodiments and variations of those embodiments.

1. A face mask that is configured to deliver pressurised gas to a user comprising:
an airbag support structure having a flexible airbag support sheet that is connected to one or more head straps;
a supply tube that is configured in use on a user's face to deliver pressurised gas from a flow generator to the face mask and is connected to the flexible airbag support sheet and an airbag, wherein the airbag is configured to induce skin sealing contact pressures from the pressurised air during use;
the airbag is further connected to one or more of the following: a nasal sealing structure of the face mask and a mouth sealing structure of the face mask;
one or more tensioner structures that are configured to augment the skin sealing pressures between the user's air-sealing skin contact regions and the face mask during use and the one or more tensioner structures are external to the airbag;
wherein the one or more tensioner structures consists of two or more elastic tensioner bands or elastic tensioner sheets, attached and stretched between attachment points or lines on the airbag on either side of where the airbag contacts the face and attachment points or lines on either the one or more head straps or the airbag support structure;
wherein the airbag is configured in use to permit freedom of movement of the airbag support structure in response to dislodging forces on the face mask; and
wherein the face mask is configured in use to maintain sealing with a user's face and to provide pressurised gas to the user's mouth, the user's nostrils or combinations thereof.

2. The face mask according to claim 1, wherein the one or more tensioner structures consists of two or more elastic tensioner bands or elastic tensioner sheets, attached and stretched between points on the airbag on either side of where the airbag contacts the face and points on either the one or more head straps or the airbag support structure that are distant from the mouth and nearer to the ears.

3. The face mask according to claim 1, wherein the airbag support structure is configured such that in use the airbag is substantially decoupled from movement of the airbag support structure from the user's facial contact surfaces.

4. The face mask according to claim 1, wherein the one or more head straps are substantially non-elastic.

5. The face mask according to claim 1, wherein the airbag is configured to fit between a portion of the user's face and the flexible airbag support sheet and provides pressurised gas to the nose and mouth of the user while allowing movement of the flexible airbag support sheet without substantially transferring the movement to the user's facial contact surfaces.

6. The face mask according to claim 1, wherein the flexible airbag support sheet further includes one or more cheek stiffeners that are configured to minimise movement when dislodging loads are experienced by the air supply tube and the flexible airbag support sheet.

7. The face mask according to claim 1, wherein the airbag is configured in use to form a substantially airtight seal with the user's face.

8. The face mask according to claim 1, wherein the airbag is configured in to use to have minimal air leakage with the user's face in contact with the mouth seal, nasal seal and/or airbag.

9. The face mask according to claim 1, wherein the airbag is configured in use to provide an adequate air seal at the skin contact regions around the mouth and/or nostrils of the user.

10. The face mask according to claim 1, wherein the face mask is configured such that in use pressurised air inflates the flexible airbag causing the airbag to push the flexible airbag support sheet and a mouth band away from each other thereby inducing tension in the one or more head straps.

11. The face mask according to claim 1, wherein the flexible airbag support sheet cooperates with the one or more head straps to transfer the dislodging forces to the user's head in a way that avoids dislodging the sealing surfaces near the mouth and/or nostrils.

12. The face mask according to claim 1, wherein the flexible airbag support sheet is non-elastic or substantially non-elastic.

13. The face mask according to claim 1, wherein the flexible airbag support sheet is elastic.

14. The face mask according to claim 1, wherein the airbag is non-elastic.

15. The face mask according to claim 1, wherein the airbag is elastic.

16. The face mask according to claim 1, wherein the airbag has a skirt region and the skirt region of the airbag is made from a floppy material that does not pass, or does not substantially pass, dislodging loads and movements experienced by the end of the supply tube to the skin contact sealing regions.

17. The face mask according to claim 1, wherein the one or more tensioner structures are two flexible and broad tensioner sheets attaching on either side of the mouth band to each of the corresponding stiffener arms.

18. The face mask according to claim 1, wherein a face seal structure is a flexible rope-like structure that is configured to be encircle the user's mouth, the user's nostrils or combinations thereof.

19. The face mask according to claim 1, wherein the airbag is constructed from a fabric that is airtight, or substantially airtight.

20. The face mask according to claim 1, wherein the airbag is configured to billow upon the addition of pressurised gas from the flow generator.

21. The face mask according to claim 1, wherein the airbag is flexible.

22. The face mask according to claim 1, wherein the one or more tensioner structures have one or more tensioner support points or lines that are configured to remain relatively stable as dislodging loads are experienced by the face mask.

23. The face mask according to claim 1, wherein the one or more head straps and/or the airbag support structure are configured to cooperate in use to provide relatively stable support to the one or more tensioner structures whereby dislodging loads and movements of the face mask are largely isolated from the one or more tensioner support structures.

24. The face mask according to claim 1, wherein the one or more tensioner structures is attached to one or more of the following; an airbag, one or more stiffener arms, the head strap system, upper cheek buckles, lower cheek buckles, outer points on the airbag support and combinations thereof.

25. The face mask according to claim 1, wherein the one or more tensioner structures are one or more tensioner sheets that are attached to one or more of the following; an airbag, one or more stiffener arms, the head strap system, upper cheek buckles, lower cheek buckles, outer points on the airbag support and combinations thereof.

26. The face mask according to claim 1, wherein the airbag is moulded with an integrated mouth seal and a nasal seal, tensioner bands and/or tensioner sheets that are one or more of the following: attached to the airbag, an integral part of the moulded airbag, or combinations thereof.
